(19) United States
(12) Patent Application Publication
(10) Pub. No.: US 2026/0340089 A1
(43) Pub. Date: Oct. 29, 2026

(54) RESILIENT MAGNETOMETER SYSTEMS WITH A MAGNETO-PUF (PHYSICAL UNCLONABLE FUNCTION)

(71) Applicants: BROWN UNIVERSITY, Providence, RI (US); NORTHERN ARIZONA UNIVERSITY, Flagstaff, AZ (US)

(72) Inventors: Gang Xiao, Providence, RI (US); Bertrand Cambou, Flagstaff, AZ (US)

(21) Appl. No.: 19/188,995

(22) Filed: Apr. 24, 2025

Related U.S. Application Data
(60) Provisional application No. 63/638,002, filed on Apr. 24, 2024.

(51) Int. Cl. G06F 21/31; A61B 5/242; G01R 33/00

(52) U.S. Cl. CPC G06F 21/31; G01R 33/0023; A61B 5/242; A61B 2562/0223; G06F 2221/2103

(57) ABSTRACT

Systems and methods are provided for implementing a challenge-response protocol. A first magnetic field source that generates a magnetic field along a first axis. A second magnetic field source generates a magnetic field along a second axis that is orthogonal to the first axis. A plurality of magnetometers each generate a first output in response to the first magnetic field and a second output in response to the second magnetic field. Differential read logic generates a signal representing a first difference between the first output of a first magnetometer of the plurality of magnetometers and the first output of a second magnetometer of the plurality of magnetometers and a second difference between the second output of the first magnetometer and the second output of the second magnetometer.

### RELATED APPLICATIONS

This application claims priority to U.S. Provisional Patent No. 63/638,002, filed Apr. 24, 2024 and entitled “Resilient Magnetometer Systems with a Magneto-PUF (Physical Unclonable Function),” which is hereby incorporated by reference in its entirety.

### TECHNICAL FIELD

This invention relates to medical imaging, and more particularly, to the implementation of a physical unclonable function using magnetometers.

### BACKGROUND

Magnetic sensors play a crucial role in various applications as they are capable of measuring the direction of the Earth's magnetic field in three dimensions, as well as electric currents, position, and orientation without contact. A common application of magnetic sensors is for position sensing, which typically employs a Hall effect sensor and a permanent magnet. Additionally, magnetic sensors have various other applications, including position tracking, navigation, anti-theft systems, vehicle detection, location of unexploded ordnance and mines, space research and geophysics, medical distance and position sensors, magnetic labeling, and non-destructive testing.

There are different types of magnetic sensors, which can be broadly classified into three categories: low sensitivity, medium sensitivity, and high sensitivity. Low sensitivity magnetic sensors are designed to measure magnetic fields stronger than Earth's and are commonly used for current measurement and magnetic memory readout. Examples of these sensors include search-coil magnetometer, Hall effect sensor, and magnetoresistive magnetometers. Medium sensitivity magnetic sensors, on the other hand, are used to measure the variation of the Earth's magnetic field. These sensors determine the orientation of the device with respect to the Earth's magnetic field and are commonly used in applications such as magnetic compasses and traffic control. Types of sensors used for these applications are search-coil magnetometers, fluxgate magnetometers, and magneto-resistive magnetometers. High sensitivity magnetic sensors are used to measure magnetic fields inside of the Earth's magnetic field due to induced or permanent dipole moments. Applications for these sensors include heart/brain scanning and metal object detection. The most common types of high sensitivity sensors include superconducting quantum interference devices (SQUID) and optically pumped magnetometers.

In real-time applications such as terminal devices and autonomous vehicles, failure of these sensors can have devastating consequences, as they are needed to operate terminal devices and autonomous vehicles in real time. For example, connected sensors are often relied upon for real-time artificial intelligence (AI) driving cyber-physical systems (CPS) with the Internet of Things (IoT). Magnetic field-based sensors are widely used, making them crucial for guiding terminal devices in real-time. Unfortunately, magnetic sensors can be vulnerable to various attack methods, such as false data injection attacks that exploit vulnerabilities in communication networks to alter sensor data. A malfunctioning sensor or a deliberate attack by a hacker can have devastating consequences.

### SUMMARY OF THE INVENTION

In one implementation, a method includes generating a challenge for a client device at a server. The challenge includes a first magnetic field strength associated with a first axis and a second magnetic field strength associated with a second axis orthogonal to the first axis. The challenge is transmitted from the server to the client device. A magnetic field defined by the first magnetic field strength and the second magnetic field strength is generated. A response representing each of a first difference between a first output of a first magnetometer, representing the first axis, and a first output of a second magnetometer, representing the first axis and a second difference between a second output of the first magnetometer, representing the second axis, and a second output of the second magnetometer, representing the second axis is generated.

In another implementation, a system includes a first magnetic field source that generates a magnetic field along a first axis. A second magnetic field source generates a magnetic field along a second axis that is orthogonal to the first axis. A plurality of magnetometers each generate a first output in response to the first magnetic field and a second output in response to the second magnetic field. Differential read logic generates a signal representing a first difference between the first output of a first magnetometer of the plurality of magnetometers and the first output of a second magnetometer of the plurality of magnetometers and a second difference between the second output of the first magnetometer and the second output of the second magnetometer.

In a further implementation, a system includes a network interface that receives a challenge from the server defining a first magnetic field strength, a second magnetic field strength, and a third magnetic field strength. A first magnetic field source generates a magnetic field with the first magnetic field strength along a first axis. A second magnetic field source generates a magnetic field with the second magnetic field strength along a second axis that is orthogonal to the first axis. A third magnetic field source generates a magnetic field with the third magnetic field strength along a third axis that is orthogonal to each of the first and second axes. A plurality of magnetometers each of the generating a first output in response to the first magnetic field, a second output in response to the second magnetic field, and a third output in response to the third magnetic field. Differential read logic generates a signal representing each of a first difference between the first output of a first magnetometer of the plurality of magnetometers and the first output of a second magnetometer of the plurality of magnetometers, a second difference between the second output of the first magnetometer and the second output of the second magnetometer, and a third difference between the third output of the first magnetometer and the third output of the second magnetometer.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 illustrates one example of a client device for use in a challenge-response protocol;

FIG. 2 illustrates one example of a system for implementing a challenge-response protocol;

FIG. 3 illustrates one example of a method for implementing a challenge-response protocol;

FIG. 4 illustrates a method for implementing a cryptographic protocol using challenge-response pairs; and

FIG. 5 is a schematic block diagram illustrating an exemplary system of hardware components capable of implementing examples of the systems and methods disclosed herein.

### DETAILED DESCRIPTION

In the context of the present disclosure, the singular forms “a,” “an” and “the” can also include the plural forms, unless the context clearly indicates otherwise. The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “substantially identical” or “substantially equal” refers to articles or metrics that are identical other than measurement error.

The systems and methods described herein use magnetometers to implement physical unclonable functions (PUFs) in a challenge-response-pair (CRP) arrangement for authentication of devices. PUFs exploit the natural variations in a manufacturing process to create unique characteristics for each device. Examples of PUFs that are currently in use include the random power-up state of Static Random Access Memory (SRAM) cells, the preformed resistance of Resistive Random Access Memory (ReRAM), and the variation of the resistance in Magnetic Random Access Memories (MRAM). Before using a PUF, a process known as enrollment is generally performed. Enrollment takes place in a secure location where a set of input signals, known as challenges, are given to the PUF device. The responses are the resulting output measurements. In the case of memory-based PUFs, the challenges are the memory locations. The ReRAM PUF, on the other hand, requires the injection of an electrical current, giving it a higher number of possible responses with increased entropy and therefore it a stronger PUF.

The set of challenges and the corresponding responses for a PUF are referred to as challenge response pairs (CRPs). For the magnetometer-based PUF disclosed herein, the challenges can include the magnitude and direction or directions of the magnetic field used to produce the response, and, where more than two sensors are used, the address of a pair of sensors to be read. The magnetic field magnitude varies continuously along each of the three axes, creating a continuous set of responses, allowing the magnetometer-based PUF disclosed herein to provide a stronger PUF than traditional digital response PUFs.

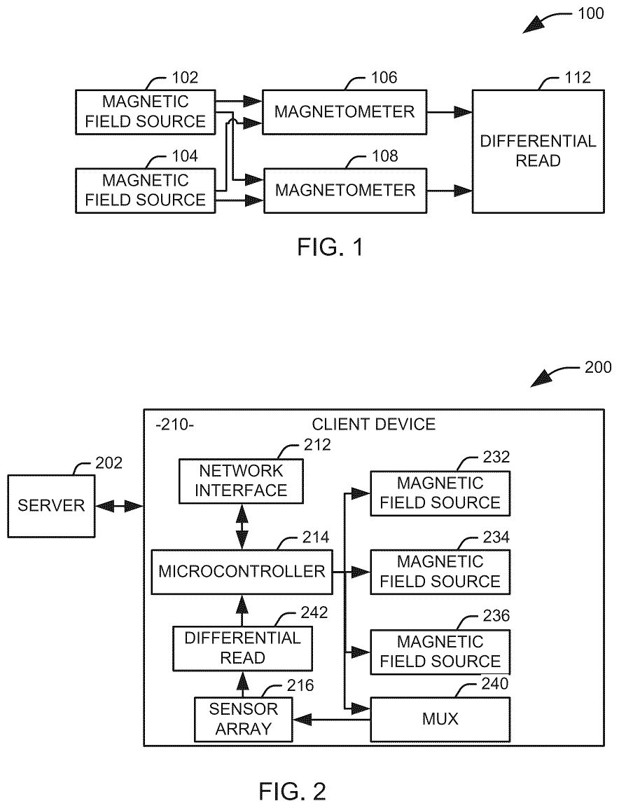

FIG. 1 illustrates one example of a client device 100 for use in a challenge-response protocol. The device includes a first magnetic field source 102 that generates a magnetic field along a first axis and a second magnetic field source 104 that generates a magnetic field along a second axis that is orthogonal to the first axis. For example, each of the magnetic field sources 102 and 104 can be implemented as planar spiral coils or soft ferromagnetic toroids that are provided with a current provided via a digital-to-analog converter. A plurality of magnetometers 106 and 108 each generate a first output in response to the first magnetic field and a second output in response to the second magnetic field. One or more of the magnetometers 106 and 108 can be calibrated, such that an expected response of the calibrated magnetometer sensor to each of a plurality of field strengths at each of a plurality of temperatures is known, and one or more magnetometers can be uncalibrated. In one example, the plurality of magnetometers and a coil associated with the first magnetic field source are implemented on a first printed circuit board and a coil associated with the second magnetic field source is implemented on a second printed circuit board that is positioned orthogonally to the first printed circuit board.

Differential read logic 112 generates a signal representing a first difference between the first output of a first magnetometer of the plurality of magnetometers and the first output of a second magnetometer of the plurality of magnetometers and a second difference between the second output of the first magnetometer and the second output of the second magnetometer. In one example, the signal is originally an analog signal, and the differential read logic 112 includes an analog-to-digital converter that converts the analog signal into a digital output. In one implementation, in which the plurality of magnetometers 106 and 108 includes more than two magnetometers, a multiplexer assembly (not shown) can selectively provides outputs from the plurality of magnetometers to the differential read logic, such that, at a given point in time, the first and second outputs of the first magnetometer and the second magnetometer are provided to the differential read logic 112 while the first and second outputs of a third magnetometer of the plurality of magnetometers are not provided to the differential read logic.

In an alternative implementation, the magnetometers 106 and 108 sense other physical parameters, such as position, orientation, or current, with the response generated in the manner described above. In these implementations, a challenge can be provided by means appropriate to the physical parameter being sensed. For example, a current can be directed through the die containing the magnetometers along a path close to the magnetometers. Low-loss rail-to-rail analog multiplexers can used to select the sensor pair to be compared.

FIG. 2 illustrates one example of a system 200 for implementing a challenge-response protocol. In the illustrated implementation, the challenge is a magnetic field, for example, defined by a three-dimensional magnetic field vector, and a selected pair of magnetometers associated with the PUF. The response generated from the PUF is a stream of bits obtained from measuring the magnetic field applied to the selected sensor pair. In one example, during an enrollment process, all possible CRPs are systematically recorded for a wide range of challenges and stored as references in the look-up table of a server. During operation, the PUF sends read-time CRPs to a server in response to challenges, and any sensor failures become immediately evident through mismatches with the reference CRPs. Cryptographic keys with high entropy can then be generated from the responses.

The system 200 includes a server 202 and a client device 210 comprising a network interface 212, a microcontroller 214, and an array of magnetometer sensors 216. In the illustrated implementation, nine magnetometers are used, but it will be appreciated that the magnetometer sensors 216 can include any type of magnetic field sensor, including Hall sensors, anisotropic magnetoresistance (AMR) sensors, giant magnetoresistance (GMR) sensors, tunneling magnetoresistance (TMR) sensors, and fluxgate sensors. Hall effect sensors produce a voltage in response to an applied magnetic field and can be used for electrical current sensing, positioning, rotation, and proximity sensing. Hall effect sensors can have either analog or digital outputs. AMR sensors, made of thin films of alloys, decrease in resistance when exposed to a perpendicular magnetic field. They are more sensitive to magnetic fields than Hall effect sensors but require separate readout electronics. Large magnetic fields can permanently alter AMR sensors. In the illustrated example, one of the magnetometer sensors is calibrated during the enrollment process, such that an expected response of the calibrated magnetometer sensor to each of a plurality of field strengths at each of a plurality of temperatures is known.

In the illustrated implementation, the client device 210 further includes three magnetic field sources 232, 234, and 236, each configured to provide a magnetic field along one of three orthogonal axes. In one implementation, the magnetic field sources 232, 234, and 236 utilize independent electric currents controlled by digital to analog converters (DACs) in the orthogonal directions on the printed circuit board (PCB) on which the magnetometer sensor array 216 is mounted. In one example, to generate a stronger magnetic field, planar spiral coils with or without soft ferromagnetic yokes can be printed or mounted on the PCB. Three pairs of orthogonal coils can be used, sized to generate a spatially uniform magnetic field vector over the magnetometers in the array 216. The DACs generate three currents through the three pairs of coils, allowing the magnetic field vector produced to be controlled by a microcontroller. It will be appreciated that the magnetic fields must be within the acceptable dynamical range of the magnetometers 216. In the illustrated example, the magnetometers can measure magnetic fields in the +/−500 μT range with a sensitivity of 0.5 μT, which is suitable for sensing the Earth's magnetic field around 50 μT. Accordingly, the injected currents should be able to vary by a factor of one thousand on each axis, thus requiring thirty bits to control the three currents in the 0.5 to 500 μT range with 0.5 μT resolution.

For generating the magnetic field component (B_{z}) in the z-direction, which is perpendicular to the PCB, planar spiral coils can be directly printed in copper on the board. For generating the magnetic field components (Bₓ and B_{y}) along the x- and y-axis, planar spiral coils can also be printed on daughter PCBs that are mutually orthogonal to the main PCB that houses the magnetometer arrays. Alternatively, a soft ferromagnetic toroid with a pair of x-coils and a pair of y-coils to generate the x-axis and y-axis components. In this implementation, the diameter of the toroid will be designed to generate spatially uniform components over the magnetometers in the array.

During operation, the client device 210 receives a challenge from the server 202 at the network interface 212 and provides it to the microcontroller 214. The challenge includes a desired magnetic field strength along each of three orthogonal axes as well as two of the magnetometer sensors 216. In one implementation, the microcontroller 214 controls the current provided to the coils generating the magnetic by controlling the voltage output of a high current gain buffer through a DAC. This allows a range of currents to be injected at different times, enabling the measurement of manufacturing differences between the sensor values over time. This voltage output generates a current through a fixed high-power resistor, which is then routed through the matched planar spiral coil pairs to produce a magnetic field having the desired strength. The microcontroller also controls a multiplexer assembly 240 that allows for the outputs of two or more of the magnetometers 216 to be selected and provided to the network interface 210. In the illustrated implementation, the multiplexer assembly 240 routes the analog voltage values from the magnetometers into the amplifiers for differential reading at differential read circuitry 242.

The differential read circuitry 242 is used to accurately read the difference between the outputs from two different sensors, converting the analog readings into a single digital signal that can be read by an ADC. In one example, the differential read circuitry 242 includes a calibration amplifier, which receives an output of the calibrated magnetometer and a differential read amplifier. The calibration amplifier can be implemented as an instrumentation amplifier whose gain is set by a resistor. The differential read amplifier takes the signal inputs from the differential outputs of the two magnetometers, allowing small difference between the two sensors to be amplified by a factor of one thousand. The signals from both the calibration and differential read amplifiers are then passed to the microcontroller, which uses them as the bits that make up a response to the challenge. Additionally, when no current is flowing through the planar spiral coil pairs, the calibrated magnetometer can be used to sense the magnetic field of the device, allowing the server to verify that the sensor is functioning correctly by comparing the reference response for the zero current case to the responses received from the remote device using the differential measurement between the sensor pair.

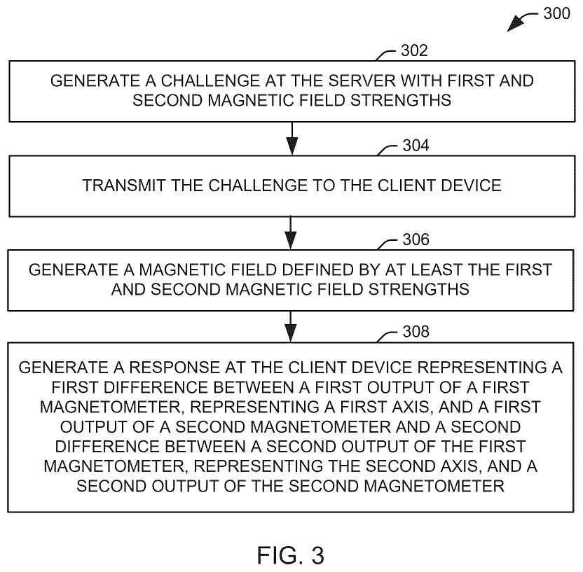

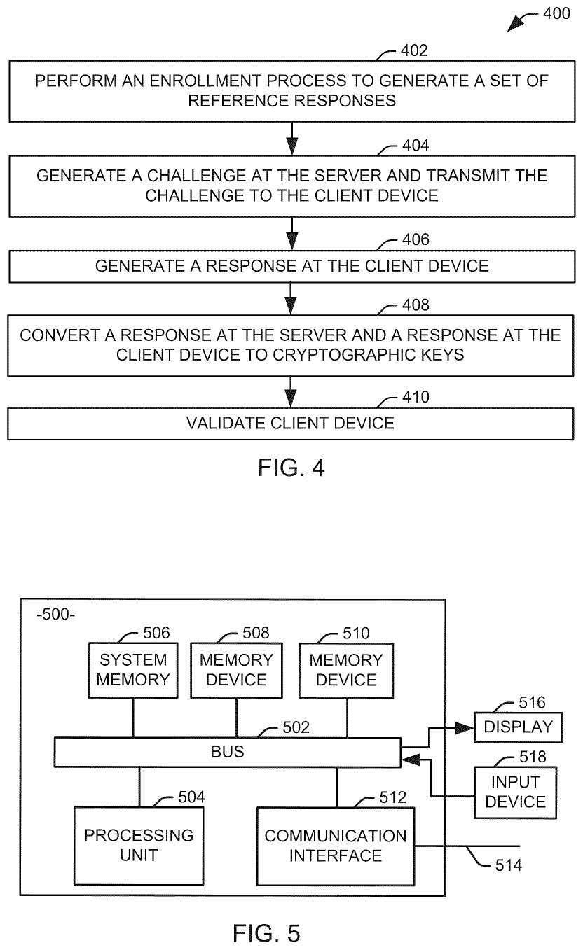

In view of the foregoing structural and functional features described above, example methods will be better appreciated with reference to FIGS. 3 and 4. While, for purposes of simplicity of explanation, the example methods of FIGS. 3 and 4 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method.

FIG. 3 illustrates one example of a method 300 for implementing a challenge-response protocol. At 302, a challenge for a client device at is generated at a server. The challenge includes a first magnetic field strength associated with a first axis and a second magnetic field strength associated with a second axis orthogonal to the first axis. In one example, in which the client device has more than two magnetometers, the challenge can also include addresses for a pair of magnetometers for evaluation. The challenge is transmitted from the server to the client device at 304. Further, a first magnetic field strength can be provided, representing an axis orthogonal to each of the first and second axes. At 306, a magnetic field defined by at least the first magnetic field strength and the second magnetic field strength is generated. It will be appreciated that the strength of the field can be controlled by varying a level of current provided to coils providing the magnetic field.

At 308, a response is generated representing each of a first difference between a first output of a first magnetometer, representing the first axis, and a first output of a second magnetometer and a second difference between a second output of the first magnetometer, representing the second axis, and a second output of the second magnetometer. Essentially, the response includes the difference in the voltage output between the magnetometer pair in response to the magnetic field along each axis. It will be appreciated that the challenge can include multiple sets of magnetic field strengths and selected pairs of magnetometers, and steps 306 and 308 can be repeated to generate additional differences between the selected pairs to append to the response.

During operation, the server and the client device openly exchange a series of challenges over a non-secure network. Based on the challenges, the server generates a sequence of reference responses using a look up table, and the client device simultaneously measures a sequence of real-time responses. There are several protocols utilizing the challenge response pairs that can be employed. For example, the server can be authenticated at the client device using a reference response provided to the terminal device. Alternatively, the client device can transmit the response to the server for comparison to a reference response. In another example, two-way secure authentication can be generated by having each of the server and the client device convert their respective response into a cryptographic key that is then used to encrypt an authentication message. The encrypted message and the necessary data to correct the cryptographic keys can be exchanged over the non-secure network. For example, each of the server and the client device can convert their respective response sequences into a symmetrical cryptographic key that is then used to encrypt messages between them. Alternatively, each of the server and the client device can convert their respective response sequences into data streams, referred to as seeds, which are used to generate public/private key pairs for asymmetrical cryptography. These key pairs can be used for various applications such as public key infrastructure (PKI), digital signatures, key encapsulation, and encrypting digital files.

FIG. 4 illustrates a method 400 for implementing a cryptographic protocol using challenge-response pairs. At 402, an enrollment process is performed on the client device to generate a set of expected responses, referred to as referred responses, for the server in preparation for authentication, key generation, and monitoring operations. During enrollment, all possible challenge-response pairs (CRPs) are systematically measured multiple times using the triaxial magnetometer array under a variety of challenges and temperatures. In the illustrated implementation, the CRPs are organized and stored as references responses in the server's look-up table. Electric current circulating in the coils used to generate the magnetic field is gradually increased to cover a desired range of magnetic field strengths along each axis. The number of increments can be reduced due to the expected linearity between the nearby vectors. A model can be used to extrapolate between measured points. For example, twelve measurements can be sufficient if the current on each coil must be measured between 1 μA and 2 mA, taken at (2^{k}) μA with kϵ{0 to 11}. The measurements are repeated multiple times along the three axes to capture both the median values and standard deviations. The outputs of the magnetometers are converted into digital signals using error-avoiding techniques, such as Gray codes, to minimize the number of bad bits. In one example, assuming a measurement accuracy of twelve bits for an analog-to-digital converter used to generate twelve levels of electric current on each of the three axes, four levels of temperatures, and nine magnetometers, the lookup table would require approximately two hundred kilobytes to store both median values and standard deviations for the response.

To address the vast number of possible challenge-response-pairs, extrapolation models can be used to compute the reference response for a specific challenge at a specific temperature. The look-up table stores values that are close to the magnetic field values and temperature for each pair of magnetometers, which are then fed into the extrapolation model to generate the reference response, which can be compared to any read-time response measured at the client device. A quicker alternative is to simply locate the closest value in the look up table and retrieve the reference response without additional calculations, which is sufficient for many applications, especially when real-time measurements are noisy and have lower precision. This simplified method also results in lower latency.

A t 404, a challenge is generated at the server and transmitted to the client device. For most cryptographic protocols, the first step in the challenge generation process is to select a random stream that can be generated by a random number generator. In the illustrated method, random streams are transformed into sequences of three-axis magnetic field vectors and addresses within the magneto-PUF that point to pairs of three-dimensional magnetometers within an array of triaxial magnetometers. To increase system entropy, the design can include M total magnetometers, where M is an integer greater than two, with (M−1) uncalibrated triaxial magnetometers in addition to one calibrated magnetometer. Both calibrated and uncalibrated magnetometers are driven by X-axis and Y-axis multiplexers (MUXs) to allow for dynamic selection of the magnetometer outputs. The number of possible pairs is equal to M(M−1). For example, if M=9, there are seventy-two available pairs. The positions of the magnetometers, Sᵢ and Sⱼ, are selected randomly with: “i” and “j” belonging to the set {1 to M} and with the condition “i≠j”. To determine a pair of magnetometers, a total of log₂(M−1) bits are needed. In the case of nine magnetometers, this results in eight bits.

The physical unclonable function generates its output by measuring the difference in readings between a pair of triaxial magnetometers exposed to a magnetic field vector. For each measurement cycle, the system requires the following minimum information a three-dimensional vector representing magnetic field strength and the address of a magnetometer pair. To increase entropy, a protocol could perform a sequence of N measurement cycles, for example, by selecting a random number, receiving a password from a user, and concatenating the random number with the password. The resulting stream is hashed and expanded with an extended output function (XoF) and organized into a sequence of N sub-streams, each able to drive a three-dimensional magnetic field for a pair of magnetometers at the client device. An example sub-stream could contain thirty bits for the three-dimensional magnetic field plus eight bits to select a pair of magnetometers. For a series of 256 measurements, the stream of challenges should then be 38×256=9728 bits long. Occasionally, in this protocol, the address of a magnetometer pair will show up twice in a pair. Such pairs are ignored, and additional sub-streams can be prepared as a precaution to replace any such pairs.

At 406, a response is generated at the client device as a stream of bits driven in real-time by the incoming challenges. In one implementation, only one of the triaxial magnetometers is calibrated, while the others are uncalibrated. The role of the calibrated magnetometer is to accurately sense the magnetic field and to be part of the group of magnetometers used to generate the response. The unique outputs of each uncalibrated sensor stem from small physical variations during sensor fabrication. The differential measurement between two uncalibrated sensors of a pair is a unique response that varies with different magnetic field strengths and temperatures. The same principle applies to a pair containing a calibrated sensor and an uncalibrated sensor. For each challenge, the response is given by the difference in output voltage between the two selected sensors along the three orthogonal axes. The differences in voltage are converted into a stream with an analog to digital converter (ADC).

To generate the portion of the response associated with each substream, currents are applied to the three orthogonal coils to get the magnetic field defined in the substream. Similarly, two multiplexers are driven to access the selected pair of magnetometers. The differential voltages between the selected pair of magnetometers are measured and converted into a digital stream at an analog-to-digital converter (ADC). The size of the digital stream is limited by the accuracy of the measurement and the precision of the ADC. By using error avoiding techniques such as Gray codes, small deviations due to noise have only limited impact on the bit error rates. The process is iterated N times to generate the sequence of responses from the N challenges. Further accuracy is achieved by monitoring the temperature and the average of the uncalibrated magnetometers to monitor the calibrated sensor.

At 408, each of the server and the client device convert their respective responses into a cryptographic key that is then used to encrypt an authentication message. For example, each of the server and the client device can convert their respective response sequences into a symmetrical cryptographic key that is then used to encrypt messages between them. Alternatively, each of the server and the client device can convert their respective response sequences into data streams, referred to as seeds, which are used to generate public/private key pairs for asymmetrical cryptography. These key pairs can be used for various applications such as public key infrastructure (PKI), digital signatures, key encapsulation, and encrypting digital files.

At 410, the client device is validated to ensure that the magnetometers are operating properly. The purpose of the magneto-PUF is to enhance system resilience by mitigating the impact of unexpected failures or malicious attacks on the calibrated sensor. The testing can be performed in multiple ways. In one example, a magnetic field is applied and the output of the calibrated magnetometer is measured and compared to a reference response. This simple challenge-response-pair mechanism can quickly verify the proper functioning of the magnetometer. In another example, the calibrated sensor is paired with one of the non-calibrated sensors in real-time. Two measurements are taken from the pair, specifically the output of the calibrated magnetometer and the difference in the outputs of the two magnetometers. The output of the calibrated sensor allows the system to determine the applied magnetic field vector, which is part of the challenge. The difference between the two magnetometers represents the response, which can be compared to the appropriate reference response, which is calculated based on the information stored in the look-up table. If the response does not match the reference response, the measurement of the magnetic field vector becomes suspect, and other magnetometers in the magneto-PUF array should be checked. Sensor failures are immediately evident as mismatches between the challenge-response-pairs. A further example uses two uncalibrated magnetometers. The process is similar to the previous example that utilizes both calibrated and uncalibrated sensors. The system analyzes the readings of two measurements, one of which provides the challenge, which is the applied magnetic field vector. The difference between readings of the two magnetometers is also the response, which can be compared to the reference response. Any discrepancies between the CRPs indicate possible sensor failures, making them immediately noticeable.

FIG. 5 is a schematic block diagram illustrating an exemplary system 500 of hardware components capable of implementing examples of the systems and methods disclosed herein, such as the server 202 and microcontroller 214 of FIG. 2. The system 500 can include various systems and subsystems. The system 500 can be a personal computer, a laptop computer, a workstation, a computer system, an appliance, an application-specific integrated circuit (ASIC), a server, a server BladeCenter, a server farm, etc.

The system 500 can include a system bus 502, a processing unit 504, a system memory 506, memory devices 508 and 510, a communication interface 512 (e.g., a network interface), a communication link 514, a display 516 (e.g., a video screen), and an input device 518 (e.g., a keyboard, touch screen, and/or a mouse). The system bus 502 can be in communication with the processing unit 504 and the system memory 506. The additional memory devices 508 and 510, such as a hard disk drive, server, standalone database, or other non-volatile memory, can also be in communication with the system bus 502. The system bus 502 interconnects the processing unit 504, the memory devices 506-510, the communication interface 512, the display 516, and the input device 518. In some examples, the system bus 502 also interconnects an additional port (not shown), such as a universal serial bus (USB) port.

The processing unit 504 can be a computing device and can include an application-specific integrated circuit (ASIC). The processing unit 504 executes a set of instructions to implement the operations of examples disclosed herein. The processing unit can include a processing core.

The additional memory devices 506, 508, and 510 can store data, programs, instructions, database queries in text or compiled form, and any other information that may be needed to operate a computer. The memories 506, 508 and 510 can be implemented as computer-readable media (integrated or removable), such as a memory card, disk drive, compact disk (CD), or server accessible over a network. In certain examples, the memories 506, 508 and 510 can comprise text, images, video, and/or audio, portions of which can be available in formats comprehensible to human beings. Additionally or alternatively, the system 500 can access an external data source or query source through the communication interface 512, which can communicate with the system bus 502 and the communication link 514.

In operation, the system 500 can be used to implement one or more parts of a system in accordance with the present invention. Computer executable logic for implementing the diagnostic system resides on one or more of the system memory 506, and the memory devices 508 and 510 in accordance with certain examples. The processing unit 504 executes one or more computer executable instructions originating from the system memory 506 and the memory devices 508 and 510. The term “computer readable medium” as used herein refers to a medium that participates in providing instructions to the processing unit 504 for execution. This medium may be distributed across multiple discrete assemblies all operatively connected to a common processor or set of related processors.

Implementation of the techniques, blocks, steps, and means described above can be done in various ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on.

What is claimed is:

1. A method comprising:
generating a challenge for a client device at a server, the challenge comprising a first magnetic field strength associated with a first axis and a second magnetic field strength associated with a second axis orthogonal to the first axis;
transmitting the challenge from the server to the client device;
generating a magnetic field defined by the first magnetic field strength and the second magnetic field strength; and
generating a response representing each of a first difference between a first output of a first magnetometer, representing the first axis, and a first output of a second magnetometer, representing the first axis and a second difference between a second output of the first magnetometer, representing the second axis, and a second output of the second magnetometer, representing the second axis.

2. The method of claim 1, further comprising:
transmitting an expected response for the challenge from the server to the client device; and
comparing the response to the expected response for the challenge to authenticate the server.

3. The method of claim 1, further comprising transmitting the response to the server.

4. The method of claim 3, further comprising comparing the response to an expected response for the challenge stored at the server to authenticate the client device.

5. The method of claim 1, further comprising:
converting the response into a cryptographic key;
encrypting a message using the cryptographic key to provide an encrypted message; and
transmitting the encrypted message to the server,
6. The method of claim 1, wherein the first and second magnetometers are first and second magnetometers of a set of at least three magnetometers and generating the challenge comprises generating the challenge with the first magnetic field strength, the second magnetic field, and addresses for the first and second magnetometers.

7. The method of claim 6, wherein the challenge further comprises a third magnetic field strength associated with the first axis, a fourth magnetic field strength associated with second axis, and addresses representing a third magnetometer and a fourth magnetometer, the method further comprising:
generating a magnetic field defined by the third magnetic field strength and the fourth magnetic field strength;
generating each of a third difference between a first output of the third magnetometer, representing the first axis, and a first output of the fourth magnetometer, representing the first axis and a fourth difference between a second output of the third magnetometer, representing the second axis, and a second output of the fourth magnetometer, representing the second axis, the response representing each of the first difference, the second difference, the third difference, and the fourth difference.

8. A system comprising:
a first magnetic field source that generates a magnetic field along a first axis;
a second magnetic field source that generates a magnetic field along a second axis that is orthogonal to the first axis;
a plurality of magnetometers, each of the magnetometers generating a first output in response to the first magnetic field and a second output in response to the second magnetic field; and
differential read logic that generates a signal representing a first difference between the first output of a first magnetometer of the plurality of magnetometers and the first output of a second magnetometer of the plurality of magnetometers and a second difference between the second output of the first magnetometer and the second output of the second magnetometer.

9. The system of claim 8, wherein the signal is an analog signal, the differential read logic further comprising an analog-to-digital converter that converts the analog signal into a digital output.

10. The system of claim 8, the system further comprising a network interface that receives a challenge from the server defining magnetic field strengths for the first magnetic field source and the second magnetic field source.

11. The system of claim 8, further comprising a multiplexer assembly that selectively provides outputs from the plurality of magnetometers to the differential read logic, such that, at a given point in time, the first and second outputs of the first magnetometer and the second magnetometer are provided to the differential read logic while the first and second outputs of a third magnetometer of the plurality of magnetometers are not provided to the differential read logic.

12. The system of claim 11, further comprising a microcontroller that controls each of the first magnetic field source, the second magnetic field source, and the multiplexer assembly according to a received challenge from a server, the challenge comprising a first magnetic field strength associated with the first axis, a second magnetic field strength associated with the second axis, and addresses of the first magnetometer and the second magnetometer.

13. The system of claim 12, wherein the microcontroller compares the signal to a reference response provided by the server to authenticate the server.

14. The system of claim 8, wherein both the plurality of magnetometers and a coil associated with the first magnetic field source are implemented on a first printed circuit board and a coil associated with the second magnetic field source is implemented on a second printed circuit board that is positioned orthogonally to the first printed circuit board.

15. The system of claim 8, wherein at least one of the plurality of magnetometers is calibrated, such that an expected response of the calibrated magnetometer sensor to each of a plurality of field strengths at each of a plurality of temperatures is known, and at least one of the plurality of magnetometers is not calibrated.

16. A system comprising:
a network interface that receives a challenge from the server defining a first magnetic field strength, a second magnetic field strength, and a third magnetic field strength;
a first magnetic field source that generates a magnetic field with the first magnetic field strength along a first axis;
a second magnetic field source that generates a magnetic field with the second magnetic field strength along a second axis that is orthogonal to the first axis;
a third magnetic field source that generates a magnetic field with the third magnetic field strength along a third axis that is orthogonal to each of the first and second axes;
a plurality of magnetometers, each of the magnetometers generating a first output in response to the first magnetic field, a second output in response to the second magnetic field, and a third output in response to the third magnetic field; and
differential read logic that generates a signal representing each of a first difference between the first output of a first magnetometer of the plurality of magnetometers and the first output of a second magnetometer of the plurality of magnetometers, a second difference between the second output of the first magnetometer and the second output of the second magnetometer, and a third difference between the third output of the first magnetometer and the third output of the second magnetometer.

17. The system of claim 16, wherein the third magnetic field source is implemented as a digital-to-analog converter that provides a current to a soft ferromagnetic toroid.

18. The system of claim 16, wherein the first magnetic field source is implemented as a digital-to-analog converter that provides a current to a planar spiral coil.

19. The system of claim 16, further comprising a multiplexer assembly that selectively provides outputs from the plurality of magnetometers to the differential read logic, such that, at a given point in time, the first, second, and third outputs of the first magnetometer and the second magnetometer are provided to the differential read logic while the first, second, and third outputs of a third magnetometer of the plurality of magnetometers are not provided to the differential read logic.

20. The system of claim 19, further comprising a microcontroller that controls each of the first magnetic field source, the second magnetic field source, the third magnetic field source and the multiplexer assembly according to a challenge received from the server, the challenge comprising a first magnetic field strength associated with the first axis, a second magnetic field strength associated with the second axis, a third magnetic field strength associated with the third axis, and addresses of the first magnetometer and the second magnetometer in a challenge received at the system.
